(19) United States
(12) Patent Application Publication
(10) Pub. No.: US 2026/0294388 A1
(43) Pub. Date: Oct. 1, 2026

(54) RADIAL ULTRASOUND IMAGE REGISTRATION METHOD AND SYSTEM

(71) Applicant: HUIWEI MEDTECH (TAIZHOU) LTD., TAIZHOU (CN)

(72) Inventors: SHENG LEI, TAIZHOU (CN); YAOCAI HUANG, TAIZHOU (CN); SHENNAN JIANG, TAIZHOU (CN)

(21) Appl. No.: 19/093,278

(22) Filed: Mar. 28, 2025

(51) Int. Cl. A61B 8/12; A61B 8/00

(52) U.S. Cl. CPC A61B 8/12; A61B 8/4254; A61B 8/5207

(57) ABSTRACT

Provided are a radial ultrasound image registration method and system. The method includes: setting a marker based on an endoscope device housing, and obtaining ultrasound data by radial scanning of an ultrasound probe; determining an amplitude lower limit, constructing a registration condition, and traversing and screening a first data group satisfying the registration condition; identifying each group of image frame head and image frame tail in the raw ultrasound data based on positions of two adjacent first data groups, wherein data between each group of the image frame head and the image frame tail is exactly data collected by the ultrasound probe during one full turn of radial scanning; and performing ultrasound image reconstruction on the data to obtain a frame of ultrasound radial-scan sectional image. Therefore, information loss and information redundancy are avoided, and the misalignment at the junction of the frame head and the frame tail is eliminated.

### TECHNICAL FIELD

The present invention relates to the field of medical imaging equipment, and specifically to a radial ultrasound image registration method and system.

### BACKGROUND

An endoscopic ultrasound examination in which an ultrasound scanning plane is perpendicular to a long axis of an endoscope is referred to as a radial endoscopic ultrasound examination. This examination enables 360° radial scanning of body cavities, and is applicable in fields such as the digestive tract, cardiovascular system (coronary arteries), and trachea/bronchi. Compared with linear endoscopic ultrasound, radial endoscopic ultrasound has a wider scanning range, is easier to control, and features a smaller probe size and a longer scanning distance, making it widely applicable.

To simplify the structure and reduce costs, a radial ultrasound endoscope device often uses a non-encoded motor to directly or indirectly drive an ultrasound probe for rotational scanning. The raw data collected by the ultrasound probe does not contain annular spatial information. A more intuitive and vivid annular image may be obtained by reconstructing the raw data based on sampling frequency, sound velocity and other parameters. To present a complete 360° radial scan image, an acquisition time of each frame of data must be equal to a time it takes for the ultrasound probe to rotate one full turn. In other words, an acquisition speed of each frame of data must match a rotation speed of the probe.

A rotation speed of a non-encoded motor cannot achieve absolute uniformity and is susceptible to load variations, and a host computer of an endoscope device cannot obtain real-time feedback on the rotation speed of the motor. As a result, it is impossible to ensure the matching between the rotation speed and data acquisition speed of the ultrasound probe, which leads to two types of image distortions:
(1) The rotation speed of the ultrasound probe is greater than the data acquisition speed. When completing the collection of a specified frame of data, the ultrasound probe has rotated more than one full turn. As a result, after the frame of data is reconstructed into an image, More than one full turn of tissue information is displayed, rather than exactly one full turn. Since the scanning start and end points are not at the same position, there is misalignment at the junction of the beginning and the end of the image. Moreover, due to the excess data in the image, tissue features such as lesions, lymph nodes, and veins are squeezed and deformed, and the relative positions of tissue features within the image are also altered. The comparison between a normal image effect diagram and an image effect diagram containing more than one full turn of data is shown in FIG. 1 of the specification. It may be seen from FIG. 1 that a shape of the lesion is squeezed and deformed, and a relative position of the lesion in the image has changed.
(2) The rotation speed of the ultrasound probe is less than the data acquisition speed. When having collected a specified frame of data, the ultrasound probe has not yet rotated a full turn, resulting in a reconstructed image containing less than one full turn of tissue information, which leads to information loss. Meanwhile, tissue features such as lesions is stretched and deformed, and the relative positions of the lesions in the image also change. The comparison between a normal image effect diagram and an image effect diagram containing less than one full turn of data is shown in FIG. 2 of the specification. It may be seen from FIG. 2 that a shape of the lesion is stretched and deformed, and a relative position of the lesion in the image has changed.

In the prior art, there is no solution to this defect. Most developers improve a rotation accuracy of an electric motor to approximately match an ultrasound data acquisition speed, thereby reducing the mismatch and improving image quality. Consequently, the phenomenon of mismatch between scanning start and end points in an image has always existed in clinical ultrasound examinations.

### SUMMARY

Based on the problems existing in the prior art, the present invention provides a radial ultrasound image registration method and system. The specific solutions are as follows:

According to a first aspect, the present invention provides a radial ultrasound image registration method, which includes:
setting a marker based on an endoscope device housing, the marker being within a scanning range of an ultrasound probe, and obtaining raw ultrasound data recording echo of the marker by radial scanning of the ultrasound probe;
determining an amplitude lower limit according to a difference between an ultrasound echo amplitude range of the endoscope device housing and an ultrasound echo amplitude range of a marker echo area;
establishing a registration condition based on the amplitude lower limit, traversing all data groups in the raw ultrasound data positioned in the marker echo area, and screening out a data group satisfying the registration condition as a first data group;
identifying each group of image frame head and image frame tail in the raw ultrasound data based on positions of two adjacent first data groups, and judging data between each group of the image frame head and the image frame tail as data collected by the ultrasound probe during one full turn of radial scanning; and
performing ultrasound image reconstruction on the data between each group of the image frame head and the image frame tail to obtain a frame of ultrasound radial-scan sectional image.

In a specific embodiment, the marker echo area is obtained by setting a marker on an inner side or an outer side of the endoscope device housing;
a position of the marker echo area and an ultrasound echo amplitude are recorded; and
based on a difference in the ultrasound echo amplitude between the endoscope device housing and the marker, the marker echo area is identified.

In a specific embodiment, a low-echo filling medium is preset between the endoscope device housing and the ultrasound probe to reduce attenuation of acoustic signals; and
an ultrasound echo amplitude of the filling medium is far less than that of the marker and that of the endoscope device housing.

In a specific embodiment, the registration condition includes:
an average value of ultrasound echo amplitudes of the data groups in the marker echo area is greater than the amplitude lower limit; and
a ratio of the amplitude lower limit to any natural number greater than or equal to 1 constitutes a lower limit score, and an average value of data groups on two sides of the marker echo area is less than the lower limit score.

In a specific embodiment, the marker echo area is an array W of x rows and y columns;
a data group satisfying the registration condition is searched by traversing the raw ultrasound data;
there is a data group Z of z1 rows and z2 columns, z1<x and z2<y, if the data group Z satisfies:
condition 1: an average value average_value of echo amplitudes of the data group is greater than an amplitude lower limit m;
condition 2: an average value of a data group W1 of x1 rows and y1 columns at a left side of the marker echo area is less than m/p, and an average value of a data group W2 of x2 rows and y2 columns at a right side of the marker echo area is less than m/p;
wherein x1≤x, y1≤y, x2≤x, y2≤y, and p≥1;
the data group Z is contained within the marker echo area and belongs to the first data group.
a row and column size of the data group is set, wherein the row and column size is less than a quantity of rows and columns of an array corresponding to the marker echo area;
the raw ultrasound data is divided into a plurality of data groups and sorted, and the first data group is searched according to a sequence number of the data groups.

In a specific embodiment, a data volume collected by the ultrasound probe during one full turn of radial scanning is converted into a quantity of data groups, so as to obtain a quantity of self-increment columns, wherein the quantity of self-increment columns is not less than a quantity of columns in the marker echo area; and
after each traversal to the first data group is completed, the sequence number of the data groups is increased by the quantity of self-increment columns in the quantity of columns to obtain a starting data group for a next traversal.

According to a second aspect, the present invention provides a radial ultrasound image registration system, which is used to implement the image registration method according to any one of the descriptions of the first aspect; wherein the image registration system includes an endoscope device;
the endoscope device includes an ultrasound probe, an endoscope device housing, a marker, and a filling medium;
the ultrasound probe is positioned in the endoscope device housing, and the filling medium is contained between the ultrasound probe and the endoscope device housing;
the filling medium is used to reduce attenuation of acoustic signals;
the marker is positioned on the endoscope device housing and is positioned in a scanning range of the ultrasound probe; and
the ultrasound probe performs radial scanning to obtain raw ultrasound data recorded with a marker echo area.

In a specific embodiment, the marker is positioned inside or outside the endoscope device housing; and
an ultrasound echo amplitude of the marker is far greater than that of the filling medium.

In a specific embodiment, the filling medium is a low-echo material in a form of a solid state, a liquid state or a gaseous state; and
an ultrasound echo amplitude of the filling medium is far less than that of the endoscope device housing.

### Beneficial Effects

The present invention provides a radial ultrasound image registration method and system, the imaging registration precision of a radial ultrasound endoscope device is improved by arranging a marker on the endoscope device, a data group satisfying a registration condition in a marker echo area is selected as an image frame head and an image frame tail, and information contained in each frame of image is data collected by the ultrasound probe during one full turn of radial scanning, so that information loss and redundant information are avoided, the mismatch between scanning start and end points in the radial ultrasound image is effectively solved, the misalignment at the junction of the frame head and the frame tail is eliminated, and the deformation of tissue features such as lesions in the image and the change of the relative position of the lesions are improved.

To make the above objectives, features and advantages of the present invention comprehensible, preferred embodiments with reference to the accompanying drawings are described in detail below.

### BRIEF DESCRIPTION OF DRAWINGS

To more clearly illustrate the technical solutions of embodiments of the present invention, the drawings required in the embodiments will be briefly described below. It should be understood that the following drawings only illustrate some embodiments of the present invention and therefore should not be considered as limitations of the scope, and for those of ordinary skill in the art, other related drawings can be obtained according to these drawings without creative efforts.

FIG. 1 is an image effect diagram of the present invention with more than one full turn of scanning;

FIG. 2 is an image effect diagram of the present invention with less than one full turn of scanning;

FIG. 3 is a schematic diagram of a flow chart of an image registration method according to the present invention;

FIG. 4 is a schematic diagram of a structure of an endoscope device according to the present invention;

FIG. 5 is a distribution diagram of ultrasound echo amplitudes of a marker, an endoscope device housing, and a filling medium according to the present invention;

FIG. 6 is an exemplary diagram of a positional relationship among data groups according to the present invention; and

FIG. 7 is a schematic diagram of a flow chart of a registration principle according to the present invention.

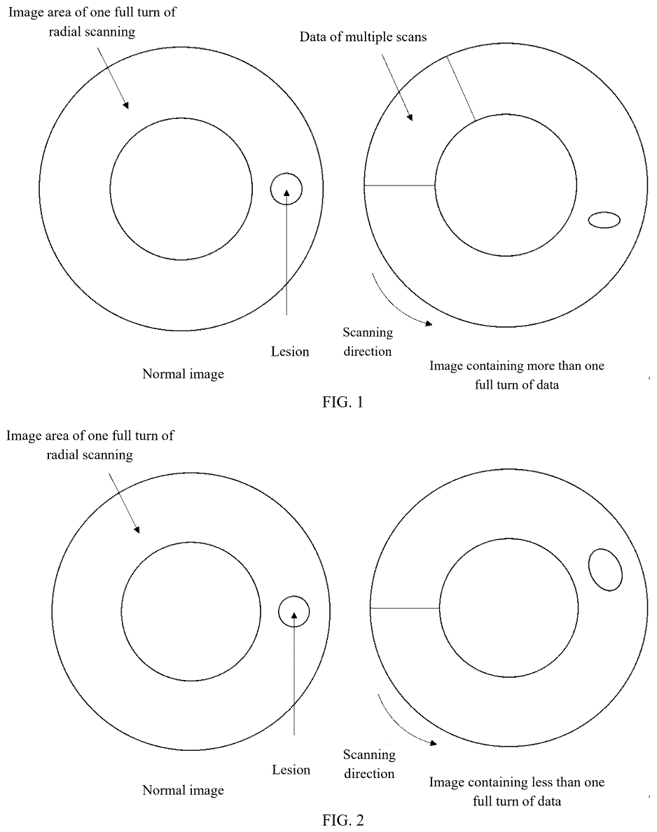

Reference numerals are as follows: 1: marker; 2: ultrasound probe; 3: filling medium; and 4: endoscope device housing.

### DETAILED DESCRIPTION OF EMBODIMENTS

The following clearly and completely describes the technical solutions in the embodiments of the present invention with reference to drawings in the embodiments of the present invention. It is clear that the described embodiments are merely a part rather than all of the embodiments of the present invention. All other embodiments obtained by those of ordinary skill in the art based on embodiments of the present invention without creative efforts shall fall within the protection scope of the present invention.

The reconstruction of an image from a radial ultrasound endoscope device requires that a frame head and a frame tail are finally fixed at the same position. If the data of the frame head and the data of the frame tail are collected from different positions of a lumen, due to the differences in the hierarchical structures of different lumen positions, there is obvious misalignment, that is, mismatching, at the junction of the frame head and the frame tail. The mismatching not only causes obvious misalignment at the junction of the frame head and frame tail, but also causes distortion of tissue features and lesion shapes in the image. In real-time image display, the mismatching also causes changes in the lesion area in the image, that is, the position of the same lesion in the continuously displayed images is inconsistent, which makes it difficult to read and diagnose image information.

The radial scanning in the present invention refers to scanning by a probe in a form of rotating collection. One full turn of radial scanning is a 360° rotation. In the data obtained by scanning, column data may represent main image data. Therefore, the reconstruction of the ultrasound data mainly involves the reconstruction of the column data in the ultrasound data.

### Embodiment 1

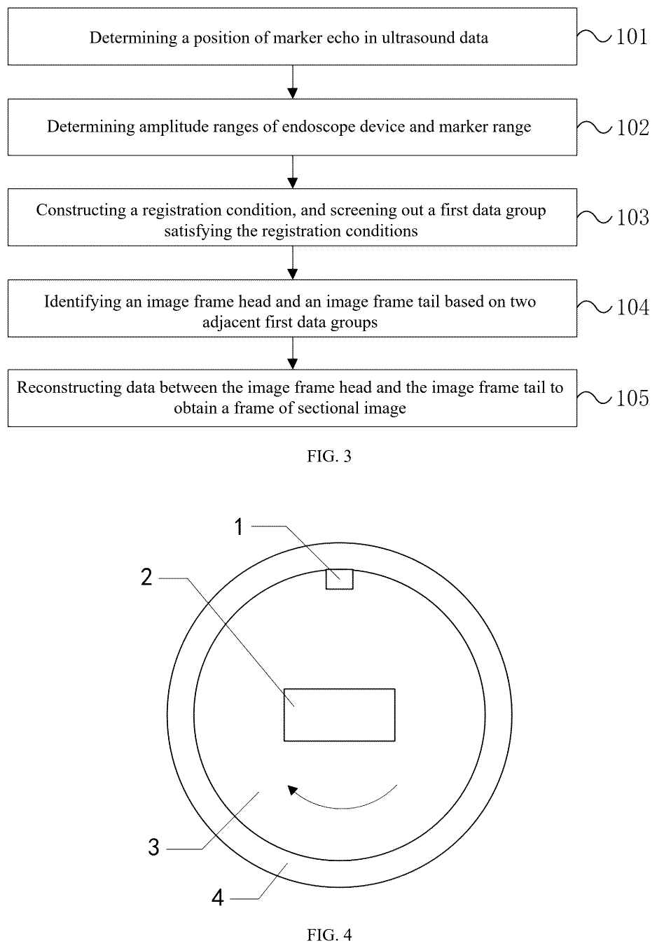

This embodiment proposes a radial ultrasound image registration method, which solves the mismatch between scanning start and end points in an image obtained by a radial ultrasound endoscope device by using a marker echo area. A flow chart of an image registration method is shown in FIG. 3 of the specification, and the specific solution is as follows:

A radial ultrasound image registration method includes the following steps:
101. setting a marker echo area based on an endoscope device housing, wherein the marker echo area is positioned in a scanning range of an ultrasound probe, obtaining raw ultrasound data recorded with the marker echo area by radial scanning of the ultrasound probe, and determining an axial position of marker echo in the ultrasound data;
102. determining an amplitude lower limit according to a difference between an ultrasound echo amplitude range of the endoscope device housing and an ultrasound echo amplitude range of a marker echo area;
103. establishing a registration condition based on the amplitude lower limit, traversing all data groups positioned at the marker echo area in the raw ultrasound data, and screening out a data group satisfying the registration condition as a first data group;
104. identifying each group of image frame head and image frame tail in the raw ultrasound data based on positions of two adjacent first data groups, wherein data between each group of the image frame head and the image frame tail is exactly data collected by the ultrasound probe during one full turn of radial scanning; and
105. performing ultrasound image reconstruction on the data between each group of the image frame head and the image frame tail to obtain a frame of ultrasound radial-scan sectional image.

This embodiment constructs a marker echo area as a marker to accurately obtain an image frame head and an image frame tail. The marker echo area is essentially an area that is different from the endoscope device housing in terms of the ultrasound echo amplitude, and has specificity in the ultrasound image. In addition, the marker echo area may be obtained by detecting a part inherent in the imaging device and having imaging specificity. In the ultrasound data obtained by radial scanning, there are obvious differences in the marker echo area and a non-marker echo area, which are easy to distinguish. A special area is set, an image frame head and an image frame tail are selected in the special area to ensure that the frame head and frame tail can match, and the data between the frame head and frame tail is exactly the data scanned for a full turn. Preferably, the marker echo area is obtained by arranging a marker inside or outside the endoscope device housing. The ultrasound echo amplitude of the marker is different from that of the endoscope device housing, and can present specificity in the raw ultrasound data. The marker is an objective “part” of the endoscope device, which needs to be assembled (arranged) at a certain position of the endoscope device to cooperate with a corresponding algorithm to complete a desired registration function. The marker echo area is an area occupied by the ultrasound image (bright spot) of the marker.

The marker echo area is a range occupied by the marker in the ultrasound image. The marker echo area refers to an area occupied by the bright spots in the ultrasound image of the marker. If there is a bright spot in about rows 100-150 and columns 500-530, this area is called the marker echo area. The ultrasound echo amplitude refers to: after the ultrasonic wave hits an object, the object reflects part of the ultrasonic wave back, and the reflected ultrasonic signals are converted into electrical signals according to the intensity relation, and the value of the electrical signals is the ultrasound echo amplitude. The ultrasound echo amplitudes of different media vary, so that the markers can be identified by using the difference.

The marker may be positioned inside or outside the endoscope device housing, as long as the marker is positioned within the scanning range of the ultrasound probe. The radial ultrasound probe performs a 360° rotation scan, and the image information of the marker is finally recorded on the raw ultrasound data.

The raw ultrasound data may be understood as a large matrix including rows and columns. According to the solution of this embodiment, the elements in the raw ultrasound data are divided into a plurality of data groups in units of groups, and the image frame head and image frame tail are determined in units of data groups, so as to facilitate the search of the first data group. Therefore, the step 102 is followed by setting a size of rows and columns of the data group. The size of rows and columns of the data group must be less than the marker echo area. For example, the marker echo area is an array of x rows and y columns, a quantity of rows of the data group must be less than x, and a quantity of columns must be less than y. The raw ultrasound data is divided into a plurality of data groups and sorted, and the first data group is searched according to a sequence number of the data groups, so that the second round of traversing is conducted quickly after the first round of traversing is completed. There is already an order in the raw ultrasound data, such as the row and column, and the sequence number of the data group may be defined according to the row and the column of the elements after the data group is divided. A quantity of rows and columns included in the data group may be set according to specific precision requirements and efficiency requirements.

In the specification, FIG. 4 is a schematic diagram of a structure of an endoscope device when a marker is positioned inside an endoscope device housing. The marker echo area is directly determined by the size of the marker, and the marker echo area also needs to satisfy a certain condition, and cannot be too large or too small. Too large marker echo area increases the difficulty in searching the first data group later, and too small marker echo area is difficult to cover a data group. In practical applications, a plurality of data groups exist in the marker echo area, but the solution of this embodiment only needs to identify one of the data groups that meets the registration condition as the first data group. The marker echo area is also essentially a larger data group. If the marker echo area is an array W with x rows and y columns, and a data group is set with z1 row and z2 column as the standard, then z1<x and z2<y.

Specifically, a low-echo filling medium is preset between the endoscope device housing and the ultrasound probe to reduce attenuation of acoustic signals; and an ultrasound echo amplitude of the filling medium is far less than that of the marker and that of the endoscope device housing. The filling medium may be a liquid, solid or gas.

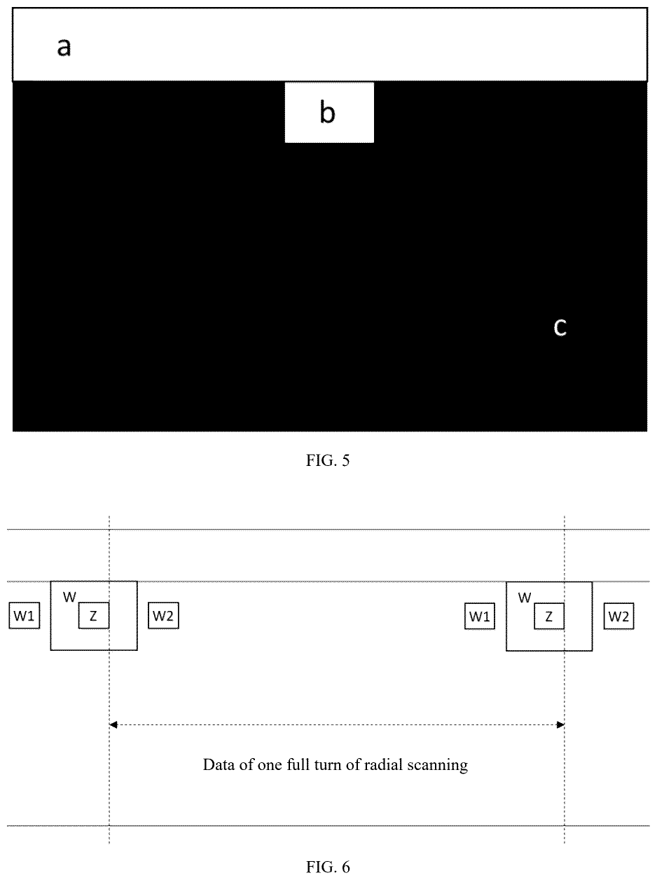

Generally, the filling medium is usually a low-echo material to reduce the attenuation of acoustic signals. Therefore, in the raw ultrasound data, the ultrasound echo amplitude of the filling medium is significantly less than that of the marker and the endoscope device housing. Assuming that the ultrasound echo amplitude of the endoscope device housing is about a, the echo amplitude of the marker is about b, and the echo amplitude of the filling medium is about c, there are a>>c (much greater), b>>c, and the distribution of the three echo amplitudes is shown in FIG. 5. The range of ultrasound echo amplitudes of the selected endoscope device housing material and marker material is always measurable, assuming m≤a≤n, m≤b≤n, and the echo amplitude c of the filling medium is significantly much less than m. m is an amplitude lower limit, and n is an amplitude upper limit. m and n are determined comprehensively based on the ultrasound echo amplitude range of the endoscope device housing and the marker echo amplitude range.

Specifically, the data groups in the marker echo area are registered, and the first data group is selected as the frame head and frame tail. The registration conditions include: the average value of the ultrasound echo amplitude of the data group in the marker echo area is greater than the amplitude lower limit; the lower limit score is constructed based on the amplitude lower limit, and the average value of the data groups at two sides outside the marker echo area is less than the lower limit score. The ratio of the amplitude lower limit to any natural number greater than or equal to 1 constitutes the lower limit score.

Assuming that the marker echo area is an array W of x rows and y columns; and a data group satisfying the registration condition is searched by traversing the raw ultrasound data;
there is a data group Z of z1 rows and z2 columns, z1<x and z2<y, if the data group Z satisfies:
condition 1: an average value average_value of echo amplitudes of the data group is greater than an amplitude lower limit m;
condition 2: an average value of a data group W1 of x1 rows and y1 columns at a left side of the marker echo area is less than m/p, and an average value of a data group W2 of x2 rows and y2 columns at a right side of the marker echo area is less than m/p; p is a constant value, and may be specifically set according to the average value of W1 and m. The average value of W1 and W2 must be less than m.
wherein x1≤x, y1≤y, x2≤x, y2≤y, and p≥1;

Then the data group Z is contained in the marker echo area, which is a part of the marker and belongs to the first data group.

The positional relationship among Z, W1 and W2 is shown in FIG. 6 of the specification.

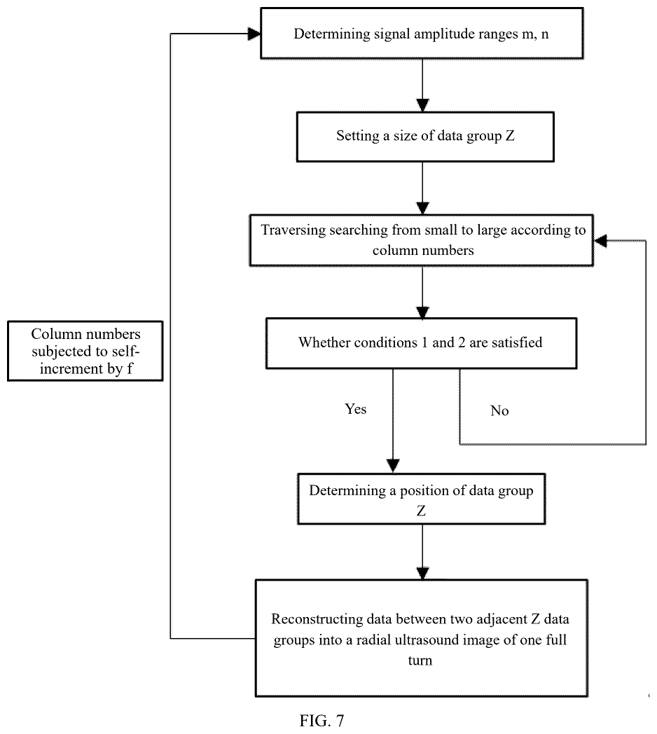

After a data group that satisfies the traversal conditions is detected, a round of traversal is completed. The traversal search range is subjected to self-increment by f (f>y), and the next data group that satisfies the traversal conditions is continued to search. The position of the detected Z data group is the frame head and frame tail of the image. The data between two adjacent Z data groups is subjected to ultrasound image reconstruction, and a series of radial ultrasound images containing a full turn of information may be obtained. The principle of the registration method is shown in FIG. 7 of the specification.

After a first data group is searched, a data volume collected by the ultrasound probe during one full turn of radial scanning is converted into a quantity of data groups, so as to obtain a quantity of self-increment columns, wherein the quantity of self-increment columns is not less than a quantity of columns in the marker echo area; and after each traversal to the first data group is completed, the sequence number of the data groups is increased by the quantity of self-increment columns in the quantity of columns to obtain a starting data group for a next traversal, and the starting data group is still positioned in the marker echo area. Since the data volume of each full turn is basically constant or in a range, the data volume that may be obtained by performing radial scanning for one full turn is preset based on an actual condition of the probe, then the data volume is converted into the quantity of data groups, a quantity of self-increment columns is obtained by integrating the factors such as errors, and the quantity of self-increment columns is increased based on the serial number of the first data group, so that the starting data group of the next round of traversal can be obtained. The starting data group is also necessarily positioned in the marker echo area, and the searching speed of the frame head and the frame tail is greatly improved.

After the first data groups are searched, the image frame head and the image frame tail are determined according to the positions of two adjacent first data groups. In FIG. 6, the data group Z is the first data group, and the image frame head also starts from the first data group, and the next adjacent first data group is the image frame tail. The data between each group of the image frame head and the image frame tail is exactly data collected by the ultrasound probe during one full turn of radial scanning, so that data loss and data redundancy are avoided, the mismatch between scanning start and end points in the radial ultrasound image is effectively solved, the misalignment at the junction of the frame head and the frame tail is eliminated, and the deformation of tissue features such as lesions in the image and the change of the relative position of the lesions are improved. Each image frame head corresponds to one image frame tail, and a frame of ultrasound array image may be obtained by reconstructing data between a group of image frame heads and image frame tails.

In this embodiment, the ultrasound probe includes a single-element ultrasound probe or an array ultrasound probe, and the image registration method is applicable to both single-element ultrasound probe radial-scanning imaging and array ultrasound probe imaging. In addition, the image registration method of the present application is applicable not only to the ultrasound endoscope device, but also to other ultrasound imaging devices that requires radial scanning.

This embodiment provides a radial ultrasound image registration method, the imaging registration precision of a radial ultrasound endoscope device is improved by arranging a marker on the endoscope device, a data group satisfying a registration condition in a marker echo area is selected as an image frame head and an image frame tail, information contained in each frame of image is data collected by the ultrasound probe during one full turn of radial scanning, so that information loss and redundant information are avoided. The mismatch between scanning start and end points in the radial ultrasound image is effectively solved, the misalignment at the junction of the frame head and the frame tail is eliminated, and the deformation of tissue features such as lesions in the image and the change of the relative position of the lesions are improved.

### Embodiment 2

This embodiment provides a radial ultrasound image registration system for implementing the image registration method of Embodiment 1. The specific solution is as follows:

The radial ultrasound image registration system, includes an endoscope device; the endoscope device includes an ultrasound probe 2, an endoscope device housing 4, a marker 1 and a filling medium 3; the ultrasound probe 2 is positioned in the endoscope device housing 4, and the filling medium 3 is contained between the ultrasound probe 2 and the endoscope device housing 4; the filling medium 3 is used to reduce attenuation of acoustic signals; and the marker 1 is positioned on the endoscope device housing 4 and is positioned in a scanning range of the ultrasound probe 2. The ultrasound probe 2 collects ultrasound data by radial scanning. The schematic diagram of the endoscope device is shown in FIG. 4 of the specification.

The marker 1 is positioned inside or outside the endoscope device housing 4; and an ultrasound echo amplitude of the marker 1 is far greater than that of the filling medium 3. The filling medium 3 is a low-echo material in a form of a solid state, a liquid state or a gaseous state; and an ultrasound echo amplitude of the filling medium 3 is far less than that of the endoscope device housing 4.

The scope of the marker 1 and the scope of the non-marker 1 in the ultrasound data obtained by the radial scanning are significantly different and may be easily distinguished. A special area is set, an image frame head and an image frame tail are selected in the special area to ensure that the frame head and frame tail can match, and the data between the frame head and frame tail is exactly the data scanned for a full turn.

The present invention provides a radial ultrasound image registration method and system, the imaging registration precision of a radial ultrasound endoscope device is improved by arranging a marker on the endoscope device, a data group satisfying a registration condition in a marker echo area is selected as an image frame head and an image frame tail, and information contained in each frame of image is data collected by the ultrasound probe during one full turn of radial scanning, so that information loss and redundant information are avoided. The mismatch between scanning start and end points in the radial ultrasound image is effectively solved, the misalignment at the junction of the frame head and the frame tail is eliminated, and the deformation of tissue features such as lesions in the image and the change of the relative position of the lesions are improved.

It should be understood by those skilled in the art that the modules of the present invention described above can be implemented by a general-purpose computing system, and can be concentrated on a single computing system or distributed over a network composed of multiple computing systems. Optionally, the modules or steps can alternatively be implemented by program codes executable by a computing system, so that the modules or steps can be stored in a storage device and executed by a computing system; or the modules or steps can be separately made into various integrated circuit modules, or multiple modules or steps thereof can be made into a single integrated circuit module for implementation. Thus, the present invention is not limited to any specific combination of hardware and software.

It should be noted that the foregoing are merely some embodiments of the present invention and applied technical principles. Those skilled in the art may understand that the present invention is not limited to specific embodiments described herein, and those skilled in the art may make various significant changes, readjustments, and replacements without departing from the protection scope of the present invention. Therefore, although the present invention is described in detail by using the foregoing embodiments, the present invention is not limited to the foregoing embodiments, and may further include more other equivalent embodiments without departing from the concept of the present invention. The scope of the present invention is determined by the scope of the appended claims.

The above disclosure is only a few specific implementation scenarios of the present disclosure, however, the present disclosure is not limited thereto. Any changes that can be thought of by those skilled in the art should fall within the protection scope of the present disclosure.

1. A radial ultrasound image registration method, comprising:
setting a marker based on an endoscope device housing, the marker being within a scanning range of an ultrasound probe, and obtaining raw ultrasound data recording echo of the marker by radial scanning of the ultrasound probe;
determining an amplitude lower limit according to a difference between an ultrasound echo amplitude range of the endoscope device housing and an ultrasound echo amplitude range of a marker echo area;
establishing a registration condition based on the amplitude lower limit, traversing all data groups in the raw ultrasound data positioned in the marker echo area, and screening out a data group satisfying the registration condition as a first data group;
identifying each group of image frame head and image frame tail in the raw ultrasound data based on positions of two adjacent first data groups, and judging data between each group of the image frame head and the image frame tail as data collected by the ultrasound probe during one full turn of radial scanning; and
performing ultrasound image reconstruction on the data between each group of the image frame head and the image frame tail to obtain a frame of ultrasound radial-scan sectional image.

2. The radial ultrasound image registration method according to claim 1, wherein the marker echo area is obtained by setting a marker on an inner side or an outer side of the endoscope device housing;
a position of the marker echo area and an ultrasound echo amplitude are recorded; and
based on a difference in the ultrasound echo amplitude between the endoscope device housing and the marker, the marker echo area is identified.

3. The radial ultrasound image registration method according to claim 2, wherein a low-echo filling medium is preset between the endoscope device housing and the ultrasound probe to reduce attenuation of acoustic signals; and
an ultrasound echo amplitude of the filling medium is far less than an ultrasound echo amplitude of the marker and an ultrasound echo amplitude of the endoscope device housing.

4. The radial ultrasound image registration method according to claim 1, wherein the registration condition comprises:
an average value of ultrasound echo amplitudes of the data groups in the marker echo area is greater than the amplitude lower limit; and
a ratio of the amplitude lower limit to any natural number greater than or equal to 1 constitutes a lower limit score, and an average value of data groups on two sides of the marker echo area is less than the lower limit score.

5. The radial ultrasound image registration method according to claim 3, wherein assuming that the marker echo area is an array W of x rows and y columns;
a data group satisfying the registration condition is searched by traversing the raw ultrasound data;
there is a data group Z of z1 rows and z2 columns, z1<x and z2<y, if the data group Z satisfies:
condition 1: an average value average_value of echo amplitudes of the data group is greater than an amplitude lower limit m;
condition 2: an average value of a data group W1 of x1 rows and y1 columns at a left side of the marker echo area is less than m/p, and an average value of a data group W2 of x2 rows and y2 columns at a right side of the marker echo area is less than m/p;
wherein x1≤x, y1≤y, x2≤x, y2≤y, and p≥1;
the data group Z is contained within the marker echo area and belongs to the first data group.

6. The radial ultrasound image registration method according to claim 1, wherein a row and column size of the data group is set, and the row and column size is less than a quantity of rows and columns of an array corresponding to the marker echo area; and
the raw ultrasound data is divided into a plurality of data groups and sorted, and the first data group is searched according to a sequence number of the data groups.

7. The radial ultrasound image registration method according to claim 1, wherein a data volume collected by the ultrasound probe during one full turn of radial scanning is converted into a quantity of data groups, so as to obtain a quantity of self-increment columns, and the quantity of self-increment columns is not less than a quantity of columns in the marker echo area; and
after each traversal to the first data group is completed, the sequence number of the data groups is increased by the quantity of self-increment columns in the quantity of columns to obtain a starting data group for a next traversal.

8. A radial ultrasound image registration system, for implementing the radial ultrasound image registration method according to claim 1; wherein the image registration system comprises an endoscope device;
the endoscope device comprises an ultrasound probe, an endoscope device housing, a marker, and a filling medium;
the ultrasound probe is positioned in the endoscope device housing, and the filling medium is contained between the ultrasound probe and the endoscope device housing;
the filling medium is used to reduce attenuation of acoustic signals;
the marker is positioned on the endoscope device housing and is positioned in a scanning range of the ultrasound probe; and
the ultrasound probe performs radial scanning to obtain raw ultrasound data recorded with a marker echo area.

9. The radial ultrasound image registration system according to claim 8, wherein the marker is positioned inside or outside the endoscope device housing; and
an ultrasound echo amplitude of the marker is far greater than an ultrasound echo amplitude of the filling medium.

10. The radial ultrasound image registration system according to claim 8, wherein the filling medium is a low-echo material in a form of a solid state, a liquid state or a gaseous state; and
an ultrasound echo amplitude of the filling medium is far less than an ultrasound echo amplitude of the endoscope device housing.
